(19) United States
(12) Patent Application Publication
(10) Pub. No.: US 2010/0323393 A1
(43) Pub. Date: Dec. 23, 2010

(54) Ordered Assembly of Membrane Proteins During Differentiation of Erythroblasts

(76) Inventors: Xiuli An, Bayside, NY (US); Mohandas Narla, New York, NY (US); Jing Liu, Woodside, NY (US); Susanne Heck, Bronx, NY (US)

Correspondence Address: K&L Gates LLP, 1900 MAIN STREET, SUITE 600, IRVINE, CA, 92614-7319, US

(21) Appl. No.: 12/820,933

(22) Filed: Jun. 22, 2010

Related U.S. Application Data
(60) Provisional application No. 61/219,700, filed on Jun. 23, 2009.

(51) Int. Cl. C12Q 1/06; C12N 5/078; C12Q 1/02; C12Q 1/04

(52) U.S. Cl. 435/39; 435/325; 435/29; 435/34

(57) ABSTRACT

Disclosed herein are methods for the isolation, identification, and quantification of red blood cells and red blood cell precursors at different developmental stages. Also disclosed are methods for monitoring ex vivo proliferation and differentiation of red blood cells and red blood cell progenitors.

### CROSS REFERENCE TO RELATED APPLICATIONS

The present application claims the benefit under 35 USC §119(e) to U.S. Provisional Patent Application 61/219,700 filed Jun. 23, 2009, the entire contents of which are incorporated by reference herein

### STATEMENT REGARDING FEDERALLY SPONSORED RESEARCH OR DEVELOPMENT

This invention was made with United States Government support of Grant Nos. DK26263, DK32094, and HL31579 awarded by the National Institutes of Health. The United States Government may have certain rights in this invention.

### FIELD OF THE INVENTION

The identification and isolation of red blood cell precursors and the use of these methods and compositions is described.

### BACKGROUND OF THE INVENTION

The study of the maturation of red cells from proerythroblast to the mature functional red blood cells (RBC) has largely been limited to the study of morphologic and cellular changes. This lack of understanding regarding the maturation process has stymied efforts directed toward understanding inherited and acquired RBC diseases such as thalassemia (Cooley's anemia), diseases affecting RBC maturation (myelodysplastic syndromes [MDS]), malarial anemia and bone marrow failure syndromes. The use of flow cytometry has helped better define each of the six stages of RBC maturation but there is much overlap and lack of clear differentiation between stages when using standard techniques.

Erythropoiesis is the process by which multipotent hematopoietic stem cells generate proliferating erythroid progenitors which subsequently undergo terminal erythroid differentiation to generate mature, non-nucleated erythrocytes. Two distinct erythroid progenitors have been functionally defined in colony assays, namely, the early-stage burst forming unit-erythroid (BFU-E) progenitor and the later stage colony forming unit (CFU-E) progenitor. The earliest morphologically recognizable erythroblast in hematopoietic tissues is the proerythroblast, which undergoes 3 to 4 mitoses to produce reticulocytes. Morphologically distinct populations of erythroblasts are produced by each successive mitosis, beginning with proerythroblasts, and followed by basophilic, polychromatic and orthochromatic erythroblasts. Finally, orthochromatic erythroblasts expel the nuclei to generate reticulocytes. Reticulocytes further mature into mature red blood cells, first in bone marrow and then in the circulation. This ordered differentiation process is accompanied by decreases in cell size, enhanced chromatin condensation, progressive hemoglobinization and marked changes in membrane organization.

During recent decades, detailed characterization of the protein composition and structural organization of the mature red cell membrane has led to insights into its function. The well studied transmembrane proteins include band 3, GPA, GPC, RhAG, Rh, CD47, Duffy, XK, Kell, CD44, Lu and LW, all of which carry blood group antigens. A two-dimensional spectrin-based skeletal network consisting of α- and β-spectrin, short actin filiments, ankyrin, protein 4.1R, adducin, dematin, tropomyosin, tropomodulin, protein 4.2 and p55 has been shown to regulate membrane elasticity and stability. Mutations in some of these proteins result in loss of mechanical integrity and hemolytic anemia. The skeletal network is attached to the lipid bilayer through two major linkages. The first is through ankyrin, which itself forms part of a complex of band 3, glycophorin A, RhAG, CD47 and ICAM-4, while the second involves protein 4.1R, glycophorin C and protein 55 with associated Duffy, XK and Rh proteins. The loss of the ankyrin-dependent linkage, due to a mutation in ankyrin or band 3, results in loss of cohesion between the bilayer and the skeletal network, leading to membrane loss by vesiculation. This diminution in surface area reduces red cell life-span with consequent anemia. A number of additional transmembrane proteins, including CD44 and Lu, have been characterized, although their structural organization in the membrane has not been fully defined.

Some transmembrane proteins exhibit multiple functions. Band 3 serves as an anion exchanger; while Rh/RhAG are probably gas transporters; and Duffy functions as a chemokine receptor. Another group of transmembrane proteins, including Lu, CD44, ICAM-4 and integrins α4β1 and α5β1 mediate cell-cell and cell-extracellular matrix interactions. CD47 prevents premature removal from the circulation by its function as a marker of “self” on the outer surface where it binds to the inhibitory receptor SIRPα. Kell possess endothin-3 converting enzyme activity.

By contrast to our broad understanding of the structure and function of the membrane of the mature red blood cells, our knowledge of the erythroblast membrane protein composition and organization in early stages is sparse. Previous studies have given evidence for asynchronous synthesis and assembly of membrane proteins, in particular α- and β-spectrin, ankyrin and band 3 during erythroid differentiation. A number of studies revealed decreased levels of expression of adhesion molecules, such as β4β1 integrin, α5β1 integrin and Lu during terminal erythroid differentiation. Finally, the transferin receptor (CD71), which is expressed at high levels in erythroblasts at all stages of development, is absent from mature red blood cells.

### SUMMARY

The present study examines the expression of red cell membrane proteins in various stages of erythroblasts derived from cells exhibiting Friend Leukemia Virus-induced anemia (FVA) by Western blot and surface expression of transmembrane proteins by flow cytometry. Distinct changes of various proteins have been observed during erythropoiesis.

Red cell membrane undergoes dramatic remodeling during erythropoiesis. However, the molecular changes during this process remain largely unknown. Twenty-four proteins in various developmental stages of erythroblasts were derived from Friend leukemia virus (FVA)-induced anemic spleen. Except for actin, which decreases during erythropoiesis, all cytoskeleton proteins were increased. The major red cell transmembrane proteins band 3, GPA, GPC, Rh, RhAG, CD47 and Duffy were only weakly expressed in proerythroblasts but were significantly increased upon differentiation. In contrast, adhesion molecules such as CD44, β1 integrin, Lu and LW were highly expressed in proerythroblasts but were lost or significantly decreased in late stage erythroblasts. Notable, the decrease in CD44 surface expression was in a progressive manner and coincided with the size change of erythroblasts. Analysis of bone marrow cells by flow cytometry using CD44 in conjunction with TER119 and forward scatter revealed six distinct populations which correspond to proerythroblasts, basophilic erythroblasts, polychromatic erythroblasts, orthochromatic erythroblasts, reticulocytes and mature red cells as confirmed by the characteristics of the sorted cells. Furthermore, sorting of bone marrow cells based on CD44 expression levels in conjunction with size change yielded nearly pure populations of different erythroblasts. Thus disclosed is an ordered assembly of membrane proteins with increased levels of proteins important for mature red cell function and decreased levels of proteins not required or even harmful for mature red cell function. Moreover, use of CD44 in conjunction of TER119 and forward scatter enabled the development of a reliable method to study erythropoiesis in vivo.

Disclosed herein are methods for isolation, identification and quantification of red blood cells at different developmental stages from mammals, including humans.

In one embodiment, disclosed herein is a method for the isolation of red blood cells (RBCS) or RBC precursors at different developmental stages comprising the steps of (a) obtaining a sample containing multiple maturation stages of RBCS or RBC precursors; and (b) isolating cells that express the cell surface marker CD44. In another embodiment, the method further comprises determining the size of the RBCS or RBC precursors.

In another embodiment, the differentiation stage of the RBC or RBC precursor is determined by CD44 expression and cell size. In another embodiment, the method further comprises the step of quantifying the number of RBCS or RBC precursors in said developmental stage.

In one embodiment, disclosed herein is a method for identifying the RBC maturation stage in a disorder of RBCS comprising the steps of (a) obtaining a sample containing multiple maturation stages of RBCS or RBC precursors; and (b) isolating cells that express the cell surface marker CD44. In another embodiment, the disorder of RBCs is selected from the group consisting of thalassemia, disorders of RBC maturation and bone marrow failure syndromes. In another embodiment, the disorder of RBC maturation is a myelodysplastic syndrome. In yet another embodiment, the thalassemia is Cooley's anemia. In another embodiment, the method further comprises the step of quantifying number of RBCs or RBC precursors in said RBC maturation stage in said disorder of RBCs.

In one embodiment, disclosed herein is a method for monitoring ex vivo proliferation and differentiation of stem cells into hematopoietic precursors and mature red cells comprising the steps of a) obtaining a sample containing multiple maturation stages of RBCs or RBC precursors; and (b) isolating cells that express the cell surface marker CD44. In another embodiment, the hematopoietic precursor is a red blood cell precursor. In another embodiment, the method further comprises the step of quantifying the number of hematopoietic precursors, RBC precursors or mature RBCs in an RBC maturation or differentiation stage.

In another embodiment, the said stem cells are from a source selected from the group consisting of peripheral blood, bone marrow, cord blood, and placenta. In yet another embodiment, the stem cells are embryonic stem cells.

### BRIEF DESCRIPTION OF THE DRAWINGS

The following drawings form part of the present specification and are included to further demonstrate certain aspects of the examples disclosed herein. The disclosure may be better understood by reference to one or more of these drawings in combination with the detailed description of specific embodiments presented herein.

FIG. 1 depicts the differentiation of Friend Leukemia Virus-induced anemia (FVA) cells in vitro. FVA cells were collected at different time points as indicated and stained.

FIG. 2 depicts immunoblots of membrane proteins in various stages of erythroblasts. FIG. 2A. Transmembrane proteins: blots of SDS-PAGE of total membrane proteins were probed with antibodies against the indicated proteins. FIG. 2B. Kell and β1 integrin after N-glycosidase treatment: 0 h or 44 h FVA cells were either untreated (−) or treated (+) with N-glycosidase and probed with anti-Kell or anti-β1 integrin antibodies. FIG. 2C. Cytoskeleton proteins: Blots of SDS-PAGE of total membrane proteins were probed with antibodies against the indicated proteins.

FIG. 3 depicts flow cytometric analysis of transmembrane proteins of different stages of FVA cells. Different stages of FVA cells were stained with antibodies against molecules as indicated. The ordinate measures the number of cells displaying the fluorescent intensity given by the abscissa.

FIG. 4 depicts flow cytometric analysis of bone marrow cells. FIGS. 4A-4C. Bone marrow cells labeled with antibodies against TER119 and CD44, FIG. 4A: plot of CD44 versus TER119; FIG. 4B: plot of CD44 versus FSC of all TER positive cells; FIG. 4C: the CD44 expression levels in the gated cell population. FIGS. 4D-4F. Bone marrow cells labeled with antibodies against TER119 and CD71

FIG. 5 depicts the isolation of different stages of erythroblasts by sorting using CD44, TER119 and FSC as markers. FIG. 5A depicts cytospin preparations of cells sorted from distinct regions as shown in FIGS. 4A and 4B were stained. FIG. 5B depicts the quantification of the purity of erythroblasts at different stages of maturation in various sorted populations using CD44.

FIG. 6 depicts a comparison of CD44 and CD71 expression in dual stained bone marrow cells. FIG. 6A depicts plot of CD44 versus FSC of all TER positive cells; FIG. 6B depicts CD44 expression levels in the gated cell population; FIG. 6C depicts CD71 expression levels in the identically gated cell populations; and FIG. 6D depicts a plot of CD44 versus CD71 of all TER positive cells.

FIG. 7 depicts the isolation of different stages of erythroblasts by sorting using CD71, TER119 and FSC as markers. FIG. 7A depicts cytospin preparations of cells sorted from distinct regions as shown in FIGS. 4A and 4B were stained. FIG. 7B depicts the quantification of the purity of erythroblasts at different stages of maturation in various sorted populations using CD71.

FIG. 8 depicts gating procedures for flow cytometry. FIG. 8A depicts all events. FIG. 8B depicts live cells (7AAD⁻ cells) gated as P1. FIG. 8C depicts a cell population within the live cells of FIG. 8B which are CD45⁻ CD11b⁻Gr1⁻(P2). FIG. 8D depicts expression of TER119 in P2 cells (P3). FIG. 8E depicts a plot of CD44⁺ vs TER119⁺ cells from P3. FIG. 8F depicts a plot of CD44⁺ vs FSC cells from P3. FIG. 8G depicts Fraction I cells dated based on CD44 and TER119 expression. FIG. 8H depicts Fractions II-IV gated based on the cluster shown in FIG. 8F. FIG. 8I depicts the distinct stages of erythroblasts sorted from bone marrow cells.

FIG. 9 depicts immunoblot (FIG. 9A) and flow cytometric (FIG. 9B) analysis of D44, GLUT1 and CD71 during in vitro human erythropoiesis. FIG. 9C depicts quantitative analysis of CD44 and GLUT1 from day 7 to day 11. FIG. 9D depicts sorted erythroblasts based on expression levels of CD44 and GLUT1.

### DETAILED DESCRIPTION OF THE INVENTION

Erythropoiesis is the process by which nucleated erythroid progenitors proliferate and differentiate to generate millions of non-nucleated red cells with their unique discoid shape and membrane material properties. The time-course of appearance of individual membrane proteins components during murine erythropoiesis throws new light on the understanding of the evolution of the unique features of the red cell membrane. Accumulation of all the major transmembrane and skeletal proteins of the mature red blood cell, except only actin, accrues progressively during terminal erythroid differentiation. At the same time, and in marked contrast, accumulation of various adhesion molecules decreases. In particular, the adhesion molecule, CD44 exhibits a progressive and dramatic decrease from proerythroblast to reticulocyte; this enabled the development of a system for distinguishing unambiguously between erythroblasts at successive developmental stages. These findings provide new insights into the genesis of red cell membrane function during erythroblast differentiation, and also offer a means of defining stage-specific defects in erythroid maturation in inherited and acquired red cell disorders and in bone marrow failure syndromes.

During terminal erythroid differentiation, proerythroblasts, the earliest morphologically recognizable nucleated erythroid cells in hematopoietic tissues, undergo 3 to 4 mitoses to generate, in humans, 2 million non-nucleated reticulocytes every second. Extensive remodeling of reticulocytes, first in the bone marrow and then in circulation results in the generation of mature circulating red cells with their unique discoid shape and membrane material properties. In the present study the temporal changes in accumulation of the different membrane proteins components during murine erythropoiesis were studied. Accumulation of all major transmembrane and all skeletal proteins of the mature red blood cell, except actin, increased progressively during differentiation from the proerythroblast to the orthochromatic erythroblast stage. In marked contrast, accumulation of a series of adhesion molecules was highest in proerythroblasts and decreased during erythroid differentiation with very low level expression in orthochromatic erythroblasts. Disclosed herein are methods which allow separation of the successive stages in erythroid differentiation with greater reliability and precision than has been previously possible.

A number of earlier studies investigated the synthesis and assembly of membrane proteins during erythropoiesis encompassing a limited number of the major membrane proteins including spectrin, ankyrin, 4.1R and band 3. In chicken erythroblasts transformed with avian erythroblastosis virus or S13 virus, expression of band 3 occurs later than that of the peripheral proteins, spectrin, ankyrin and 4.1R. Similar results on accumulation of spectrin, ankyrin, 4.1R and band 3 were also found to hold for murine and human erythropoiesis. In studies of the order of appearance of proteins that encode human blood group antigens in an in vitro culture system, Kell antigen was the first protein to be expressed.

Disclosed herein are protein expression profiles of highly synchronous erythroblast populations defining the stage-specific expression patterns of a range of proteins of the erythrocyte membrane. The accumulation of proteins involved in linking the lipid bilayer to the skeletal protein network (band 3, RhAG, ankyrin and 4.1R) follows behind that of the components of the membrane skeleton (α- and β-spectrin, adducin and tropomodulin). Therefore, the assembly of a fully functional spectrin-based network, which determines the material properties of the membrane, is a late event in erythropoiesis. In this context, it is interesting to note that the components of the spectrin-based network, α- and β-spectrin, adducin, and tropomodulin are synthesized earlier than the linking proteins, starting at the proerythroblast stage and progressively increasing at later stages of differentiation. An exception to the general pattern is actin, another principal component of the membrane skeleton, the expression of which is highest in proerythroblasts and falls off as terminal erythroid differentiation proceeds. The implication is that actin has additional function in erythroblasts, which it probably exercises in its filamentous state in the cytoplasm, whereas only a small proportion is required to form the short protofilaments of the skeletal lattice.

Erythropoiesis in vivo occurs entirely in erythroid niches, termed “erythroblastic islands”, which are made up of a central macrophage surrounded by developing erythroblasts. Adhesive interactions in this specialized structure between the central macrophage and erythroblasts, as well as between erythroblasts and extracellular matrix proteins, play a critical role in regulating terminal erythroid differentiation. A number of proteins expressed on erythroblasts, including β1 integrin, CD44, Lu and ICAM-4, are responsible for various adhesive interactions. Five splice variants of β1 integrin, arising from alternative splicing of the cytoplasmic domain designated, β1A, β1B, β1C-1, β1C-2 and β1D, have previously been identified in various cells and two of the five known isoforms are expressed during erythroid differentiation. The discovery that the adhesion molecules are most strongly expressed in proerythoblasts and are either expressed at very low levels or not at all in orthochromatic erythroblasts indicates that adhesive interactions are dynamically regulated during terminal erythroid differentiation.

The studies disclosed herein have yielded identification of a cell surface marker that discriminates between erythroblasts at different stages of maturation. The surface expression of CD44 decreased by 30-fold in a stepwise manner in passing from the proerythroblast to the orthochromatic erythroblast. The resulting ability to obtain, by cell sorting, highly purified populations of erythroblasts at all stages of maturation from primary bone marrow cells validated the choice of marker. By contrast, CD71, which has been in routine use as a surface marker for this purpose, has proved less effective CD71 expression changes only fourfold and not in a progressive manner during terminal erythroid differentiation. This lack of significant decline in CD71 is physiologically relevant since uptake of transferrin bound iron is needed for heme synthesis at all stages of erythroid differentiation to sustain high levels of hemoglobin synthesis and as such little change in its expression is to be expected.

As used herein, the term “substantially pure” refers to a population of cells that contains no more than 10% undesirable cells and can be considered 90% pure. For example, a substantially pure population of orthocromatic erythroblasts will have at least 90% orthocromatic erythroblasts by the criteria disclosed herein and less than 10% other types of cells. In additional embodiments, the substantially pure population of cells disclosed herein contains less than 8%, or less than 5% undesirable cells. Consequently these cell populations can be considered 92% pure or 95% pure, respectively.

Disclosed herein are methods useful for identifying the stage of maturation of erythrocytes and erythroblasts. As used herein, the term “red blood cell” can refer to erythrocytes, erythroblasts and reticulocytes. The present inventors have surprisingly determined that the cell surface marker CD44, optionally combined with a determination of cell size, can be used to determine the stage of differentiation or maturation of red blood cells.

In one embodiment, the methods disclosed herein are suitable for monitoring ex vivo proliferation and differentiation of erythrocyte lineage stem cells. In another embodiment, the methods used herein are suitable for monitoring in vivo proliferation and differentiation of erythrocyte lineage stem cells.

In other embodiments, the methods disclosed herein are suitable for determining the differentiation stage of red blood cells in vivo or in vitro and in normal or disease states. In

There is not limitation on the biological samples that can be used in the methods disclosed herein. In one non-limiting example, the sample is from blood, bone marrow, cord blood, placenta or spleen, Additionally, the sample can be from an in vitro or ex vivo culture of cells. In other embodiments, the sample can be from blood donors on whom apheresis has been performed in order to collect adult stem cells from the peripheral blood and/or from processing methods in which stem cells are being induced to mature into erythrocytes.

### EXAMPLES

### Example 1

### Ordered Assembly of Membrane Proteins during Differentiation of Erythroblasts

Expression of Transmembrane Proteins during Erythropoiesis.

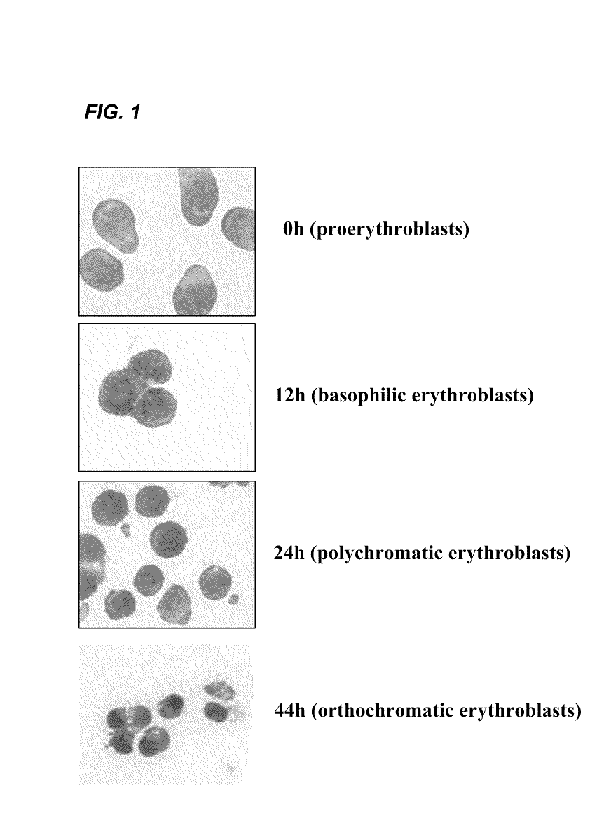

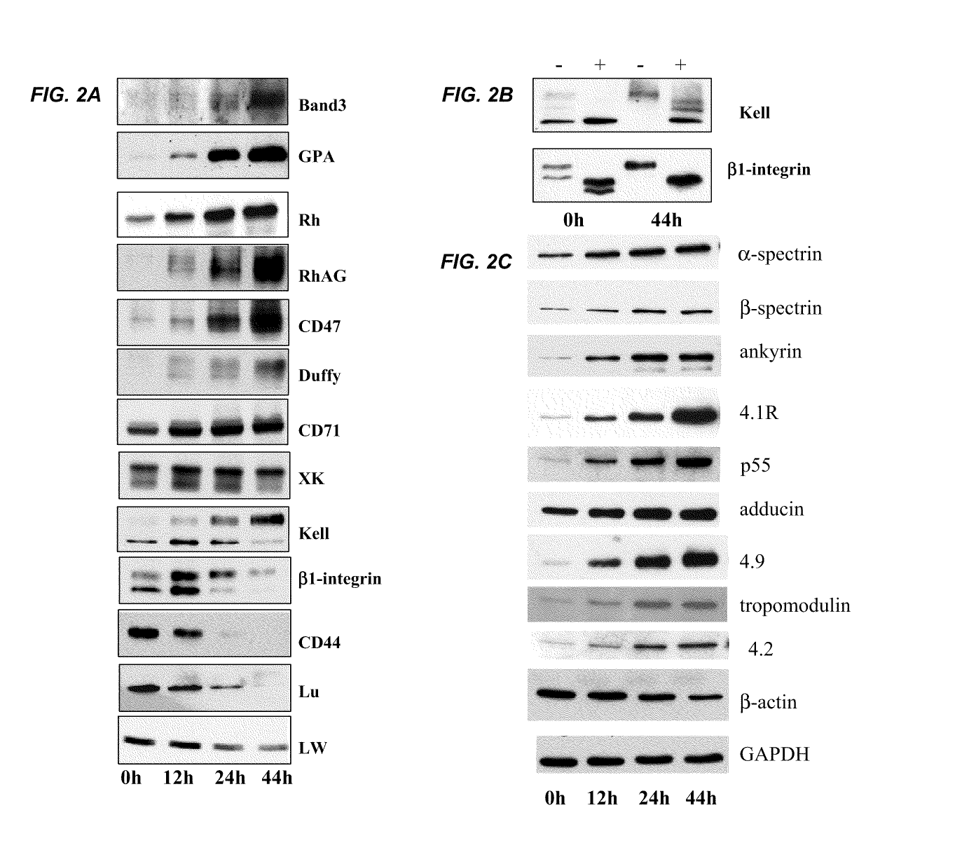

It is expected that the differentiation of erythroblasts would be accompanied by the changes in protein composition and properties of the plasma membrane, however a comprehensive study has not been reported. Using FVA system (Koury et al. J. Cell Physiol. 121:526-63, 1984) near homogenous populations of erythroblasts were obtained at proerythroblast, basophilic erythroblast, polychromatic erythroblast, orthochromatic erythroblast stages (depicted in FIG. 1) and examined the expression of 13 transmembrane proteins by Western blotting. During 44 hours of culture in this system, proerythroblasts (0 hr) progressively differentiated into basophilic erythroblasts (12 hr), polychromatic erythroblasts (24 hr) and orthochromatic erythroblasts and reticulocytes (44 hr). FIG. 2A depicts the relative concentrations of these proteins as assessed by Western blotting. It revealed following changes: 1) the major red cell proteins band 3, GPA, GPC, Rh, RhAG, Duffy and CD47 were expressed at low level in proerythroblasts but were significantly increased in late stage erythroblasts; 2) by contrary, adhesion molecules β1 integrin, CD44, Lu and ICAM-4 were expressed at the highest level in proerythroblasts and were decreased in late stage erythroblasts; 3) the transferrin receptor (CD71) and XK were slightly increased in the progression from proerythroblasts to basophilic erythroblasts.

Two distinct immunoreactive protein bands were observed for Kell and β1 integrin (FIG. 2A) and N-glycosidase treatment was performed to determine if the two bands reflect differences in glycosylation or expression of different isoforms (FIG. 2B). While this did not change the migration of the lower 94 kDa band of Kell in proerythroblasts (0 hr), the upper band (130 kDa) expressed in orthochromatic erythroblasts (44 hr) decreased in apparent size, implying that the unglycosylated 94 kDa component is expressed in proerythroblasts while the glycosylated 130 kDa component was expressed in later stages of erythroid differentiation. Two (31 integrin bands were seen in proerythrocytes (0 hr), both of which shifted after N-glycosidase treatment, implying that both are expressed at this stage and are glycosylated. Only the glycosylated higher molecular weight isoform was expressed at low levels in orthochromatic erythroblasts (44 hr).

Expression of Cytoskeleton Proteins during Erythropoiesis.

To fully understand the molecular changes of membrane protein during erythropoiesis, cytoskeletal protein compositions were also compared in various stages of erythroblasts. The expression levels of 10 skeletal proteins during terminal erythroid differentiation determined by Western blotting are shown in FIG. 2C. In contrast to the three distinct patterns of expression of transmembrane proteins, all skeletal proteins, with the exception of actin, adhered to a single pattern of expression. The expression of α-spectrin, β-spectrin, ankyrin, 4.1R, 4.2, p55, tropomodulin, dematin and adducing, increased during terminal differentiation, whereas that of actin decreased in late-stage erythroblasts compared to proerythroblasts.

Surface Exposure of Transmembrane Proteins during Erythropoiesis.

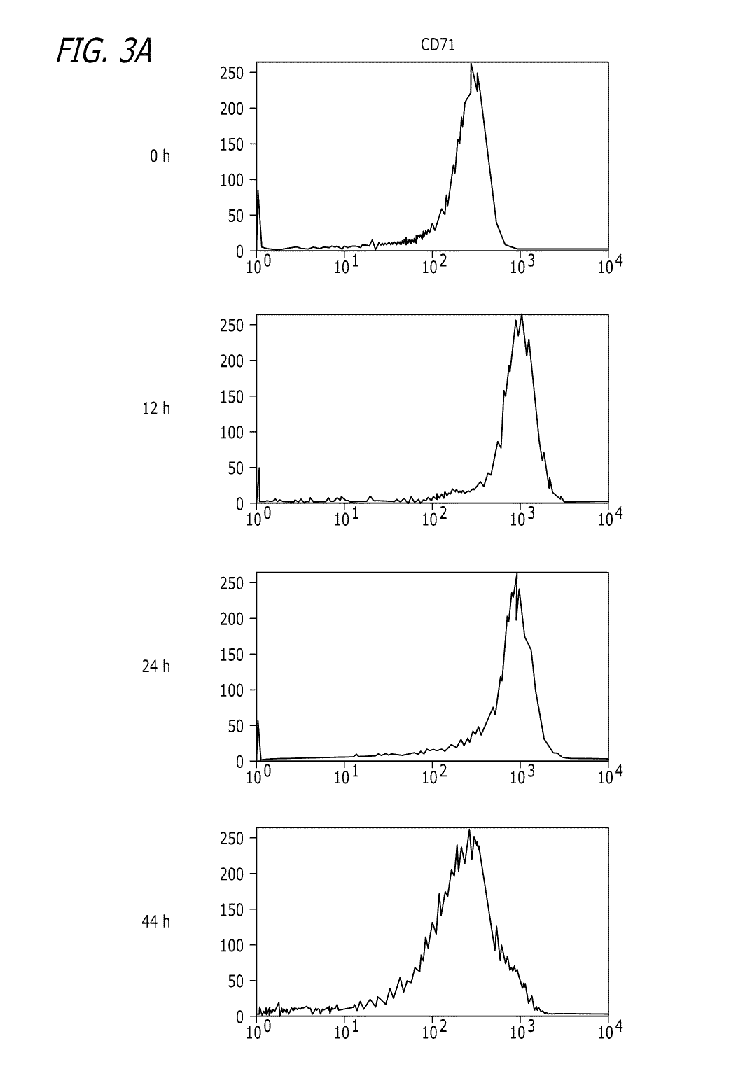

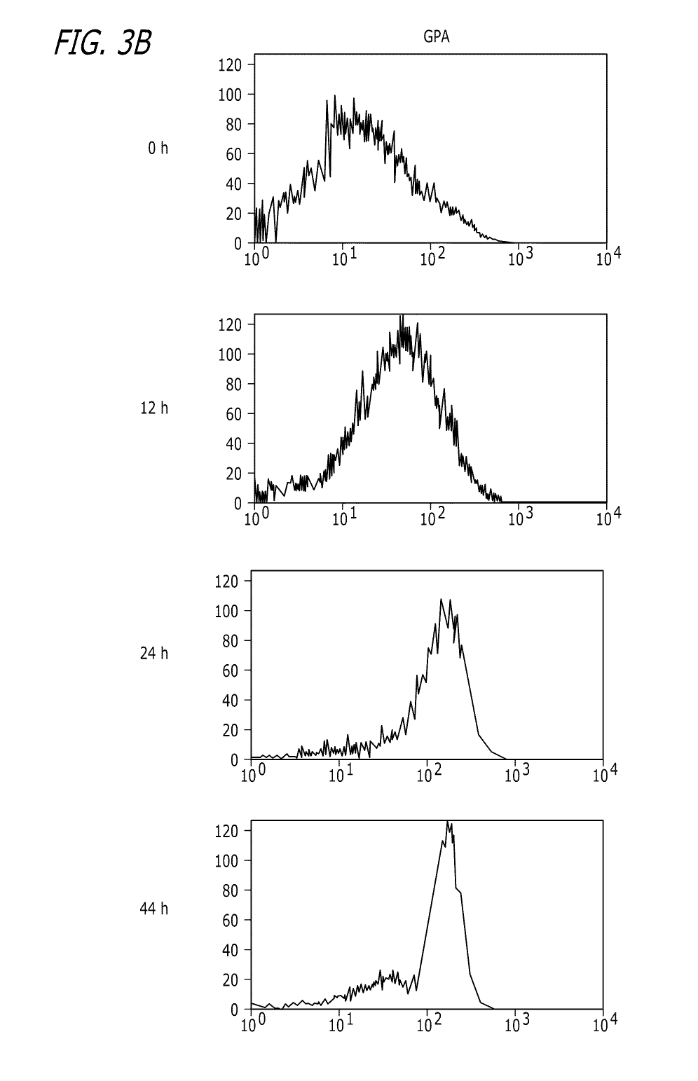

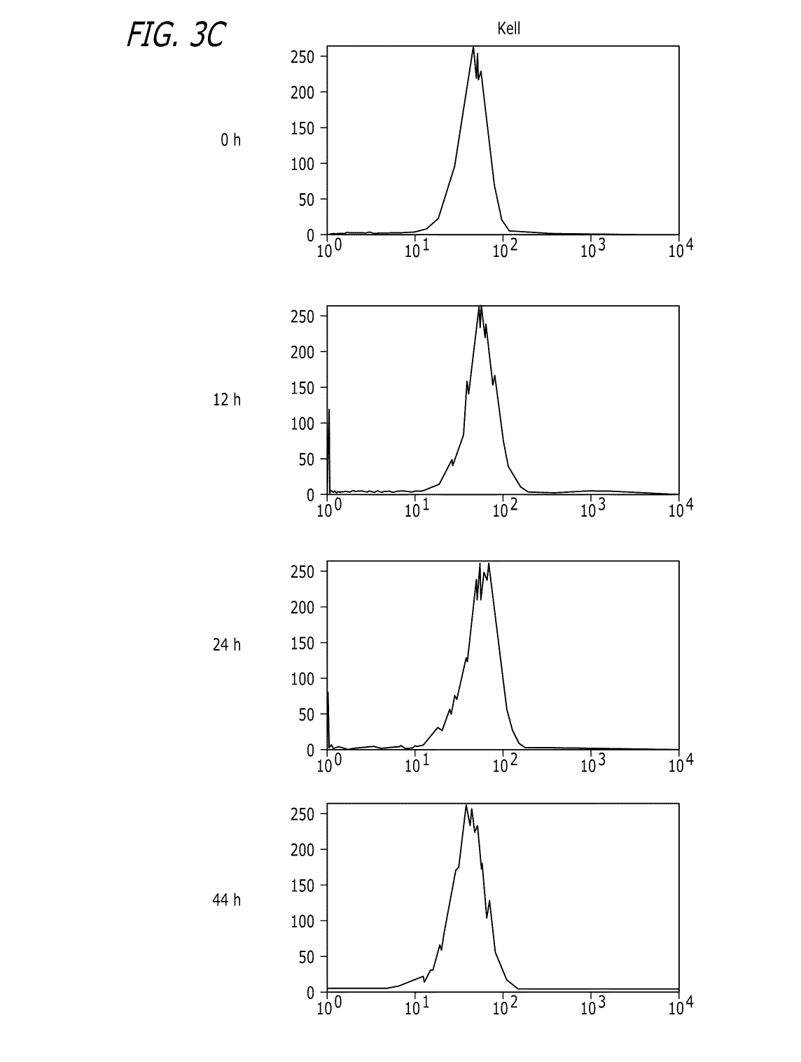

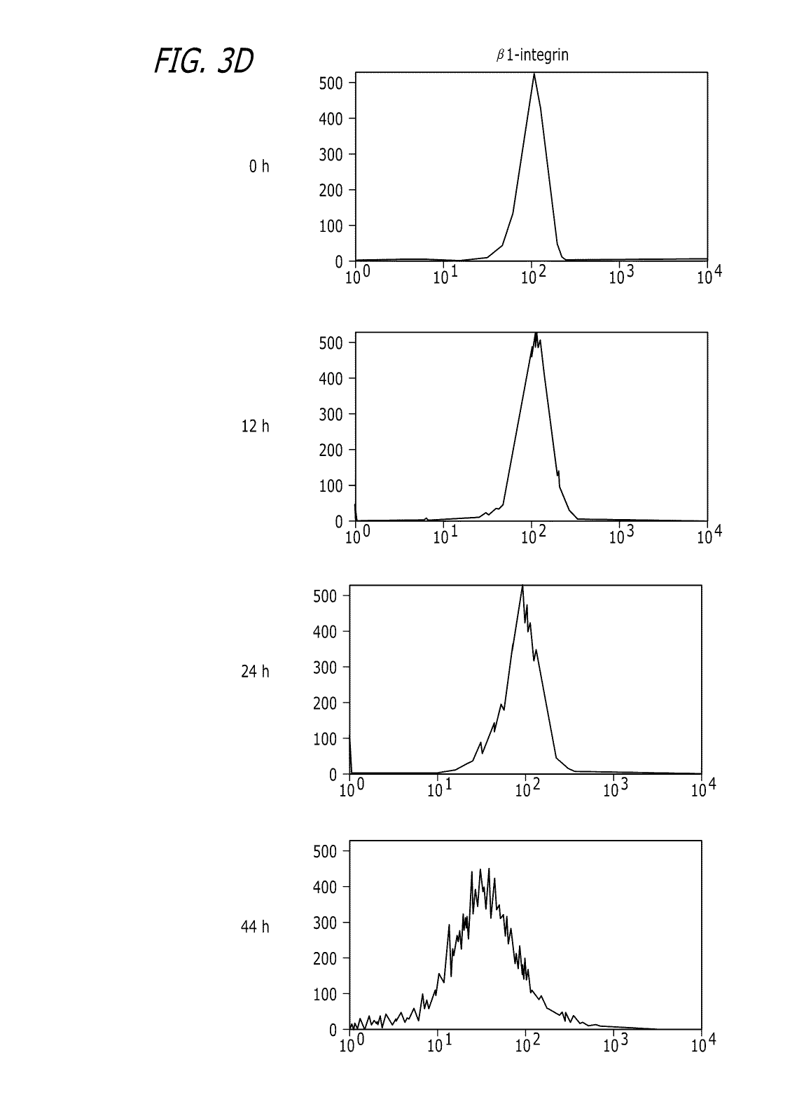

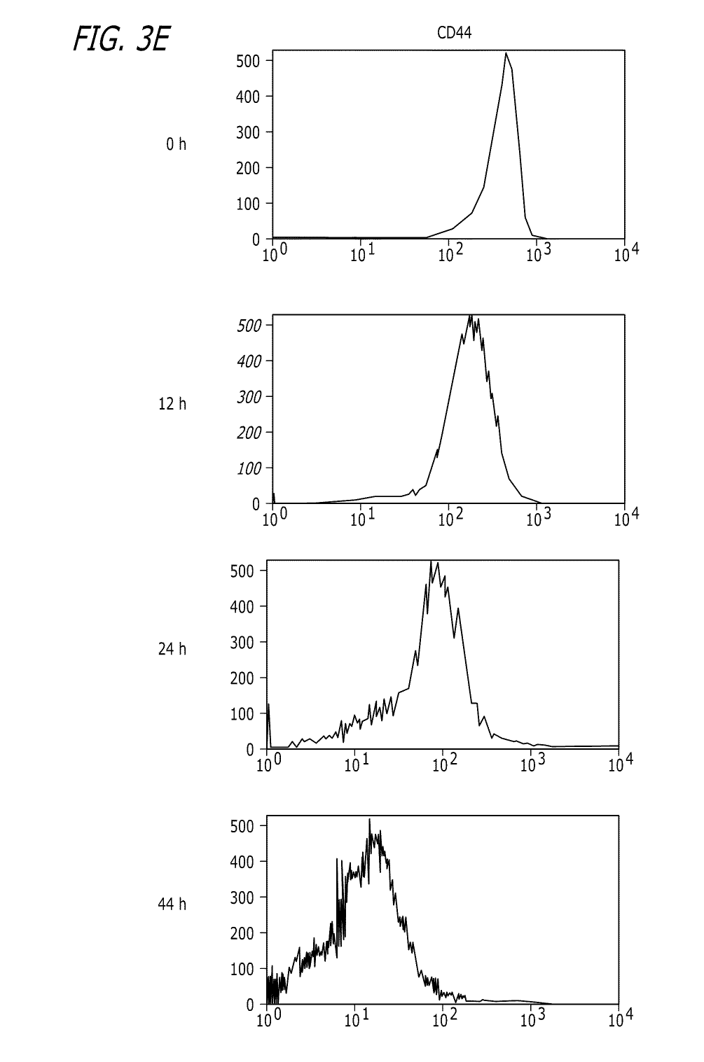

Since Western blot analysis provided total protein content, the surface expression of some transmembrane proteins including CD71, GPA, Kell, β1 integrin and CD44 was further examined by flow cytometry. As shown in FIG. 3 and Table 1, the surface expression of CD71 increased three- to four-fold from proerythroblasts to basophilic and polychromatic erythroblasts. However, late-stage orthochromatic erythroblasts expressed similar levels of surface CD71 as that of proerythroblasts. GPA exhibited a progressive increase, with four times greater abundance in orthrochromatic erythroblasts as compared to proerythroblasts. No significant change was observed for surface expression of Kell. As for β1 integrin, in contrast to the results of Western blot analysis, which showed an increase from proerythroblasts to basophilic erythroblasts, followed by a progressive decrease in late stages, surface expression of β1-integrin was significantly decreased only in orthochromatic erythroblasts. The most dramatic change occurred in surface expression of CD44, which decreased more than 30-fold from proerythroblast to othochromatic erythroblasts. The mean fluorescence intensity of unstained cells as well as cells stained with secondary antibody was less than 100.

TABLE 1
Expression of Surface Markers During Erythropoiesis

| Proteins | 0 hr | 12 hr | 24 hr | 40 hr |
|---|---|---|---|---|
| CD71 | 6700 ± 100 | 26000 ± 730 | 22000 ± 450 | 6500 ± 180 |
| GPA | 1000 ± 80 | 1800 ± 50 | 3500 ± 130 | 3700 ± 100 |
| Kell | 1300 ± 15 | 1600 ± 30 | 1600 ± 40 | 1170 ± 30 |
| β1 integrin | 2500 ± 130 | 2880 ± 180 | 2360 ± 140 | 760 ± 25 |
| CD44 | 8900 ± 400 | 4870 ± 160 | 2070 ± 40 | 270 ± 10 |

Immunofluorescence Staining of Transmembrane Proteins in Bone Marrow Erythroblasts.

The expression of GPA, band 3, RhAG, CD71 and CD44 was then examined in primary mouse bone marrow erythroblasts by immunofluorescence microscopy. Erythroblasts were defined by positive staining of GPA and DAPI. The early- and late-stage erythroblasts were distinguished based by cell size, large cells representing an early-stage, and small ones late-stage erythroblasts. As shown in FIG. 1, an increase in cell surface expression of GPA, band 3 and RhAG from early- to late-stage erythroblasts was readily apparent. In marked contrast, there was a dramatic decrease in surface expression of CD44. Little change in the surface expression of CD71 was noted between early- and late-stage erythroblasts. These findings are consistent with the results on cultured erythroblasts.

Relationship between Erythroblast Size and Expression Levels of CD44 and CD71.

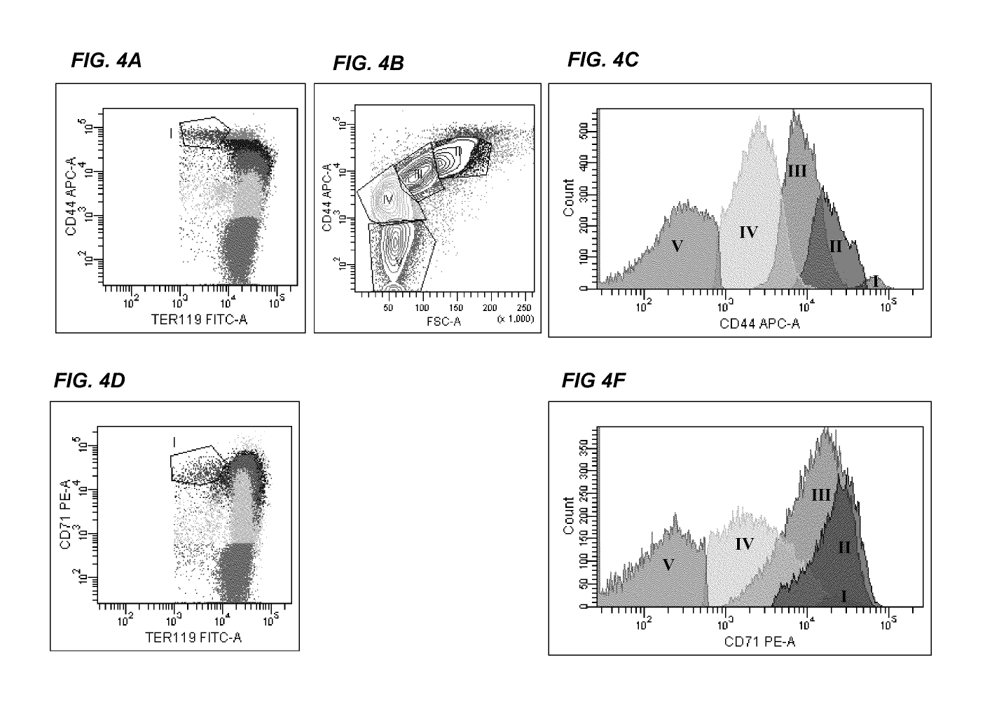

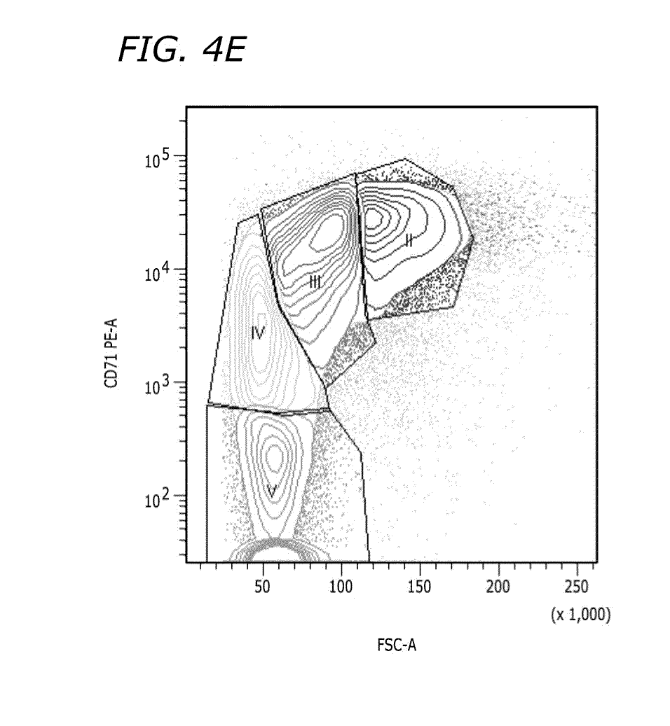

CD71 in conjunction with TER119 has been used as a surface marker to distinguish different stages of erythroblasts in vivo based on the assumption that CD71 decreases significantly during erythropoiesis. However, the present inventors have demonstrated that both total and surface expression of CD71 dose not have significant changes during erythropoiesis. Instead, the total as well as the surface expression of CD44 demonstrated a progressive reduction from stage to stage and decreased more than 30-fold from proerythroblast to orthochromatic erythroblast. These findings suggest that CD44 is a more reliable surface marker for distinguishing between different stages of erythroid differentiation than CD71. To confirm this observation, bone marrow cells were stained with both CD44 and an erythroid-specific glycophorin A antibody, TER119. FIG. 4A depicts a plot of CD44 versus TER119. Based on the TER119 staining intensity, two distinct populations, TER^{low} and TER^{hi}, were seen. The terms “low” and “hi” when used relative to cell surface expression of proteins refers to relative levels of expression. To further distinguish erythroblast subpopulations, forward scatter (FSC) intensity was used since FSC is a function of cell size and erythroblasts decrease in size with maturation. FIG. 4B depicts expression levels of CD44 as a function of FSC for all TER119 positive cells. Five distinct clusters can be seen. The histographic presentation of CD44 expression levels in the five gated cell populations (FIG. 4C) shows progressive decrease of CD44 surface expression with decreased cell size.

In parallel, bone marrow cells were also stained with TER119 and CD71 and the data analyzed in a similar manner (FIG. 4D-4F). Based on TER119 staining intensity, two distinct populations TER^{low} and TER^{hi} were once again seen (FIG. 4D). However, when CD71 expression levels were analyzed as a function of FSC for all TER⁻ positive cells (FIG. 4E), there was a marked overlap in the histogram profiles of CD71 between the gated clusters I to III, implying similar levels of CD71 (FIG. 4F).

Characterization of Erythroblast Populations by Sorting using CD44 or CD71.

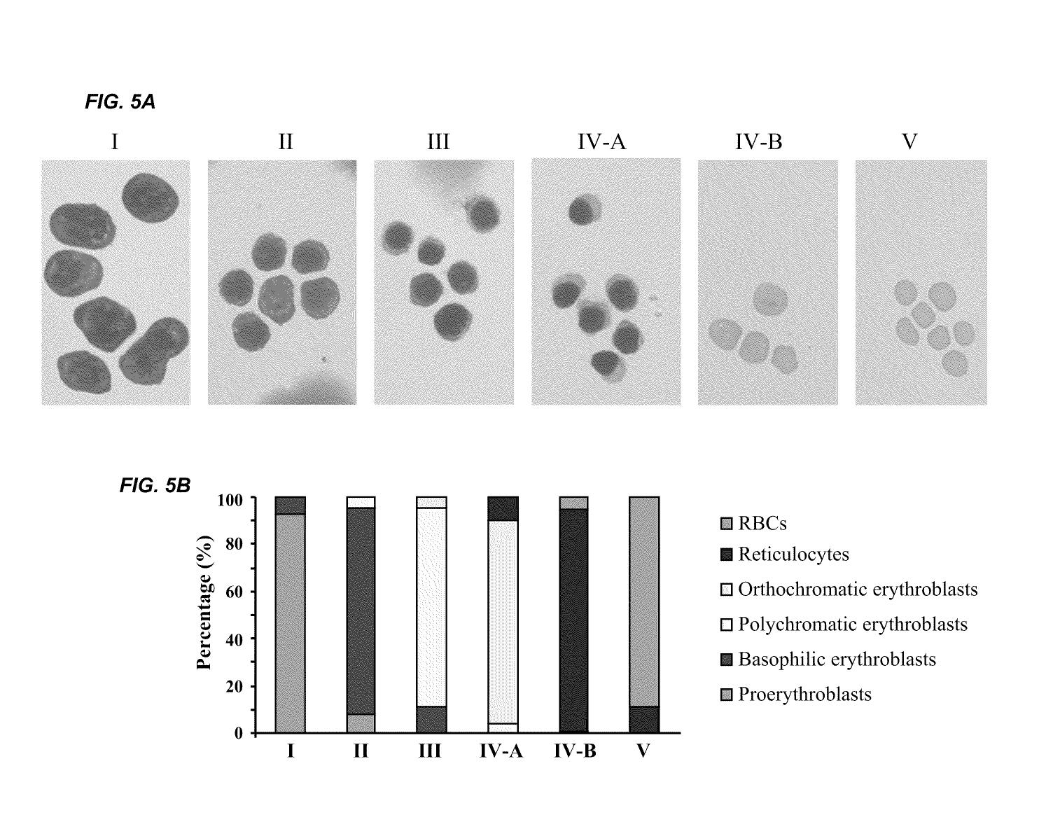

To identify different erythroblast populations, primary bone marrow erythroid cells were sorted based on either CD44 or CD71 expression levels and cell size. FIG. 5A depicts representative images from each of the five CD44 stained populations. Cells from region I have morphological characteristics of proerythroblasts, namely large size, very high nucleus/cytoplasm ration and intensely basophilic cytoplasm. Cells from region II are smaller in size, with increased nuclear condensation and the morphological characteristics of basophilic erythroblasts. Cells from region III are polychromatic erythroblasts, exhibiting the further decrease in cell size and additional nuclear condensation. Initial sorting of the region IV population showed mixed populations of orthochromatic erythroblasts and immature reticulocytes. Region iv cells were thus gated into two distinct populations based on the expression levels of CD44, termed IV-A (higher CD44 expression, top half of region IV) and IV-B (lower CD44 expression, bottom half of region IV). As shown in FIG. 5A, cells from region IV-A have cellular characteristics of orthochromatic erythroblasts while cells from region IV-B are non-nucleated reticulocytes. Finally, cells from region V were predominantly mature red cells. To quantify the purity of the various sorted populations, a differential count of erythroblasts at different stages of development was performed by examining 1000 cells. As shown in FIG. 5B, the various sorted populations contained cells at a defined stage of development ranging from proerythroblasts to reticulocytes with purities ranging from 85 to 90%.

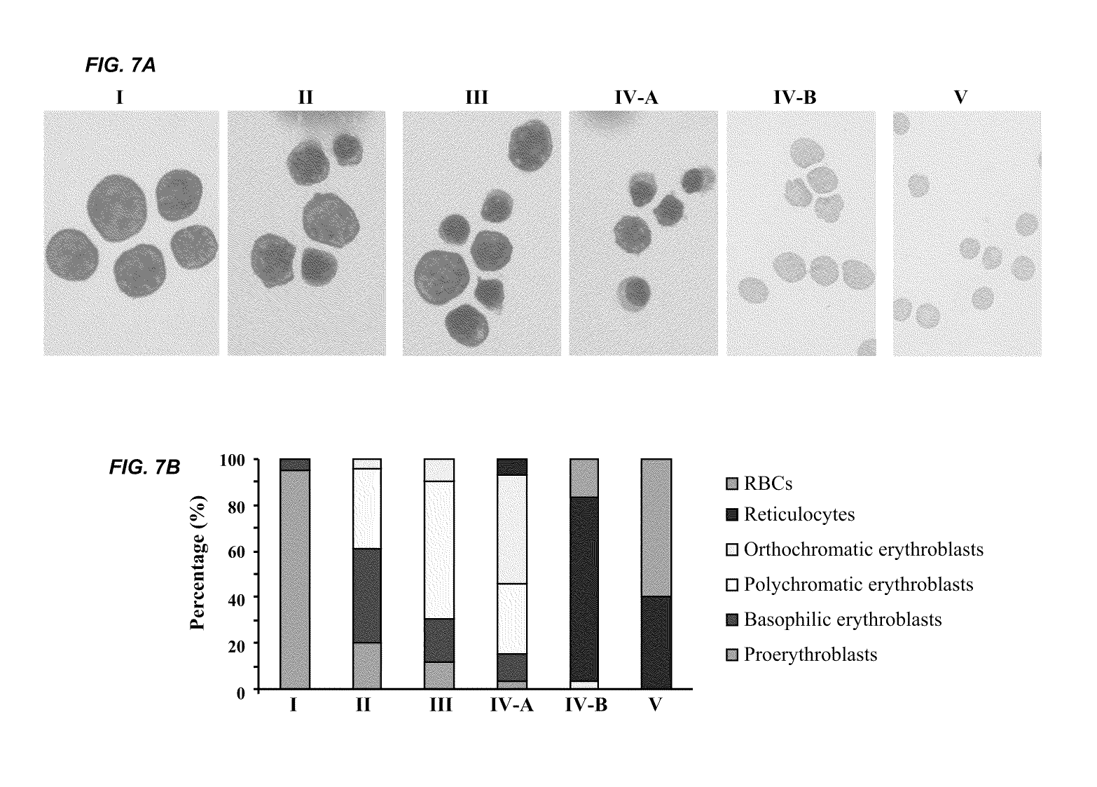

Representative images of erythroblast morphology on stained cytospins of each of the five CD71 stained populations, are shown in FIG. 7A. While, as with CD44, more than 90% of cells from region I were proerythroblasts, there was large degree of heterogeneity in all other regions (FIG. 7B). The purity of erythroblasts at all later stages of development ranged between 40 to 60% in the different fractions.

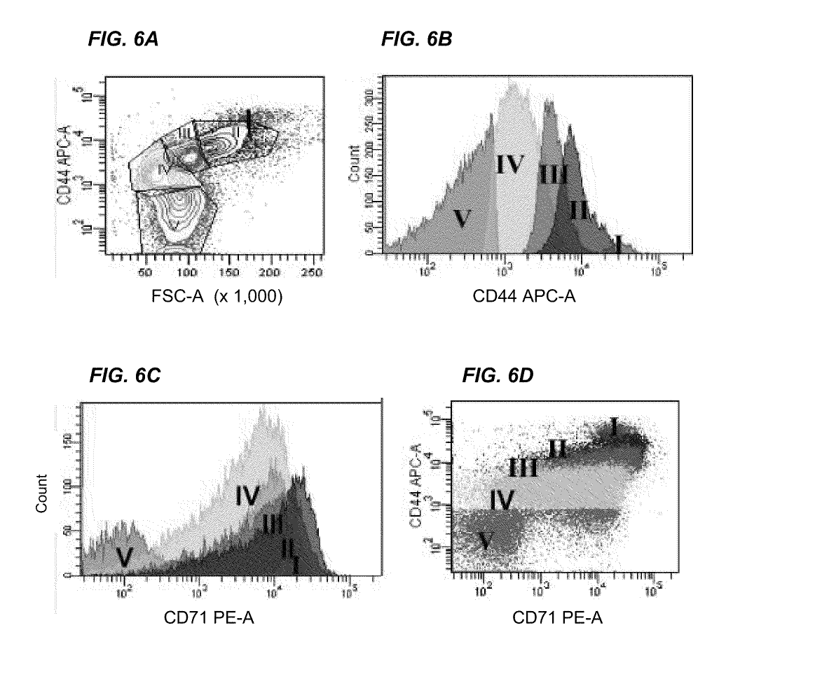

To further validate that CD44 is a more effective surface marker for distinguishing erythroblasts at different stages of erythroid differentiation than CD71, expression levels of CD44 and CD71 were compared on the same cells were measured by dual staining of primary bone marrow cells with both antibodies along with TER119. As shown in FIGS. 6A and 6B, gating on five distinct forward scatter gates of the dual stained cells, identified erythroblasts with five distinct levels of CD44 expression, consistent with staining with CD44 staining alone. In marked contrast, there was significant overlap in CD71 expression levels in the same five gated populations (FIG. 6C). Moreover, as shown in FIG. 6D, there is a wide range of CD71 expression levels at several maturation stages compared to CD44, confirming CD71 does not change progressively and distinctly during terminal erythroid differentiation.

Materials and Methods

Antibodies. For western blot, most antibodies are generated and characterized in as previously described (Salomao et al. Proc. Natl. Acad. Sci. USA 105:8026-31, 2008). Anti-TER119, anti-β1 integrin and anti-CD44 were obtained from BD Pharmingen. Anti-CD71 was from Invitrogen. For flow cytometry and sorting, the antibodies used are as following: FITC-conjugated anti-TER119, APC-conjugated anti-CD 44, PE-conjugated anti-CD71, APC-Cy™ 7-conjugated CD11b, all of which were from BD Pharmingen, FITC-conjugated anti-β1-Integrin was from BioLegend and monoclonal anti-Kell was generated internally.

Erythroid Cultures. Proerythrocytes were isolated and cultured using methods previously established (Koury et al. J. Cell PHysiol. 121:526-32, 1984; Bondurant et al. Blood 61:751-8, 1983). Briefly, two weeks after infection with 10⁴ spleen focus-forming units of the anemia-inducing strain of Friend leukemia virus (FVA), female CDF-1 mice (Charles River) were sacrificed and splenic proerythroblasts were purified by velocity sedimentation at unit gravity. The proerythroblasts were cultured at 37° C. in a humidified atmosphere of 5% CO₂ in air at a cell concentration of 1×10⁶ cells/mL in Iscoves-modified Dulbecco medium (IMDM; Gibco) with 30% heat-inactivated, fetal bovine serum (Invitrogen), 1% deionized bovine serum albumin (Millipore), 100 units/mL penicillin G (ATCC), 100 g/mL streptomycin (ATCC), 0.1 mM α-thioglycerol (Sigma), and 0.2 units/mL human recombinant erythropoietin (EPO; R&D). Erythroblasts of different stage were collected at 12 hr, 24 hr and 44 hr.

Western blot analysis. Whole-cell lysates of erythroid cells were prepared with RIPA buffer (150 mM NaCl, 1.0% Nonidet P-40, 0.5% sodium deoxycholate, 0.1% SDS, 1 mM EDTA, and 50 mM Tris HCl, pH 8.0) in the presence of protease inhibitor cocktails (Sigma). Protein concentration was measured using the DC protein assay kit (BioRad). For N-glycosidase treatment, 40 μg total protein diluted in 50 μL lysis buffer were digested with or without 5 unit N-glycosidase F (NglyF, Sigma) for 16 hr at 37° C. Thirty micrograms of protein were run on 10% SDS/PAGE gels and transferred to nitrocellulose membrane (BioRad) for 2 hr at 60V. The membrane was blocked for 1 hr in PBS containing 5% nonfat dry milk, and 0.1% Tween-20, and then probed for 2 hr with primary antibody diluted in 5% nonfat milk and 0.1% Tween-20. After several washes, blots were incubated with secondary antibody coupled to HRP (Jackson Laba) diluted in 5% nonfat milk and 0.1% Tween-20, washed, and developed on Kodak BioMax MR film (Sigma) using the SuperSignal West Pico Chemiluminescent Substrate (Thermo Scientific).

Immunofluorescence microscopy. Bone marrow cells were washed 3 times by centrifugation in PBS/0.5°)/0 BSA, and allowed to adhere for 1 hr to slides coated with Cell-Tak (BD Pharmingen). The cells were washed 3 times in PBS; fixed with 4% paraformaldehyde, 0.2% Triton X-100 in PBS at room temperature for 10 minutes; further permeabilized by 1% Triton X-100 in PBS for 3 min; and then rinsed with PBS. After incubation in 10% donkey serum (Jackson ImmunoResearch) and 10 mg/mL bovine serum albumin (BSA; Sigma) in PBS for 1 hr to block nonspecific protein binding, fixed cells were treated with primary antibodies diluted in 10 mg/mL PBS/BSA for 1 hr at room temperature. Cells were washed at 5-minute intervals 6 times with gentle shaking, incubated for 1 hr with secondary antibodies at 1:700 in 10 mg/mL PBS/BSA, washed 3 times in PBS; then mounted using Vectashield with DAPI (Vector Laboratories). Fluorescence was imaged using a Zeiss Axiovert 135 microscope with a 63×/1.25 oil immersion objective and equipped with a CCD camera.

Flow cytometric analysis of cultured cells and primary bone marrow cells. Cultured erythroblasts (1×10⁶) were stained with PE-conjugated CD71, APC-conjugated CD44, FITC-conjugated TER119, FITC-conjugated β1 integrin or unconjugated anti-Kell in PBS/0.5% BSA for 20 min at 4° C. Then the cells were washed twice in PBS/0.5% BSA. For Kell, the cells were then incubated for 20 min with goat anti-mouse IgG-FITC (Invitrogen) and the cells were washed twice in PBS/0.5% BSA and the surface antigen expression of these proteins was analyzed. Bone marrow cells were harvested from the tibia and femur of mice (3 months old). For phenotype analysis by flow cytometry, 2×10⁶ cells were re-suspended in 80 μl PBS/0.5% BSA. Cells were blocked with rat anti-mouse CD16/CD32 (5 μg/10⁶ cells) for 15 min. Then, samples were stained with FITC rat anti-mouse TER119 (1 μg/10⁶ cells), APC rat anti-mouse CD44 (1 μg/10⁶ cells) and APC-Cy™ 7 rat anti-mouse CD11b (0.3 μg/10⁶ cells) on ice for 20-30 min in the dark. Cells were washed twice with 700 μl PBS/0.5% BSA. Finally, cells were re-suspended in 100 μl PBS/0.5% BSA and stained with the viability marker 7-AAD (0.25 μg/10⁶ cells) on ice for 10 min in the dark. Cells were then suspended in 0.4 ml of PBS/0.5% BSA and analysed within one hour following staining using BD FACSDiva™ software on FACSCanto™ flowcytometer. Unstained cells or cell stained with second antibody only (in the case of Kell staining) were used as negative controls. Mean fluorescent intensity (FLI) was used as a measure of antibody binding.

Fluorescence activated cell sorting. To isolate erythroblasts at different stages of maturation by cell sorting, 200×10⁶ cells were re-suspended in 8 ml PBS/0.5% BSA in a 50 ml tube. Cells were blocked with rat anti-mouse CD16/CD32 (5 μg/10⁶ cells) for 15 min. Then, samples were stained with FITC rat anti-mouse TER119 (1 μg/10⁶ cells), APC-Cy™ 7 rat anti-mouse CD11 b (0.3 μg/10⁶ cells) and APC rat anti-mouse CD44 (1 μg/10⁶ cells) or with PE rat anti-mouse CD71 (1 μg/10⁶ cells) instead of CD44 and incubated on ice for 20-30 min in the dark. Cells were washed twice with 40 ml PBS/0.5% BSA and re-suspended in 10 ml PBS/0.5% BSA and stained with the viability marker 7-AAD (0.25 μg/10⁶ cells) on ice for 10 min in the dark. Sorting was performed on a MOFLO high speed cell sorter (Beckman-Coulter).

Cytospins. For determining cell morphology, 100 μl of cell suspension containing 10⁵ sorted cells were used to prepare cytospin preparations on coated slides using the Thermo Scientific Shandon 4 Cytospin. The slides were stained with May-Grunwald (Sigma) solution for 5 min, rinsed in Tris buffer pH7.2, for 90 sec and subsequently stained with Giemsa solution (Sigma).

### Example 2

### Quantitative Analysis of Erythropoiesis in Mouse Bone Marrow

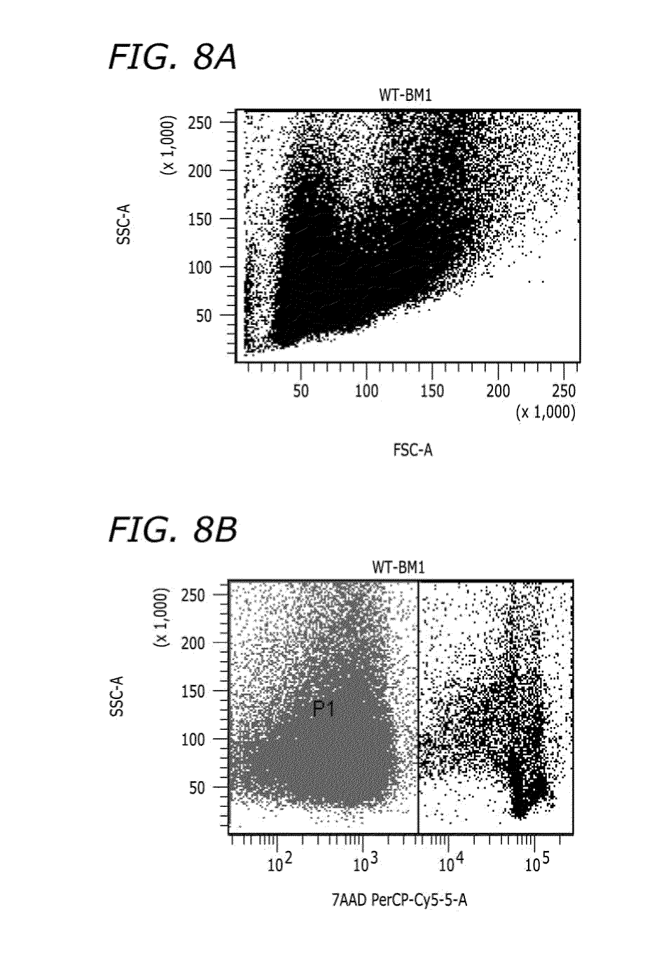

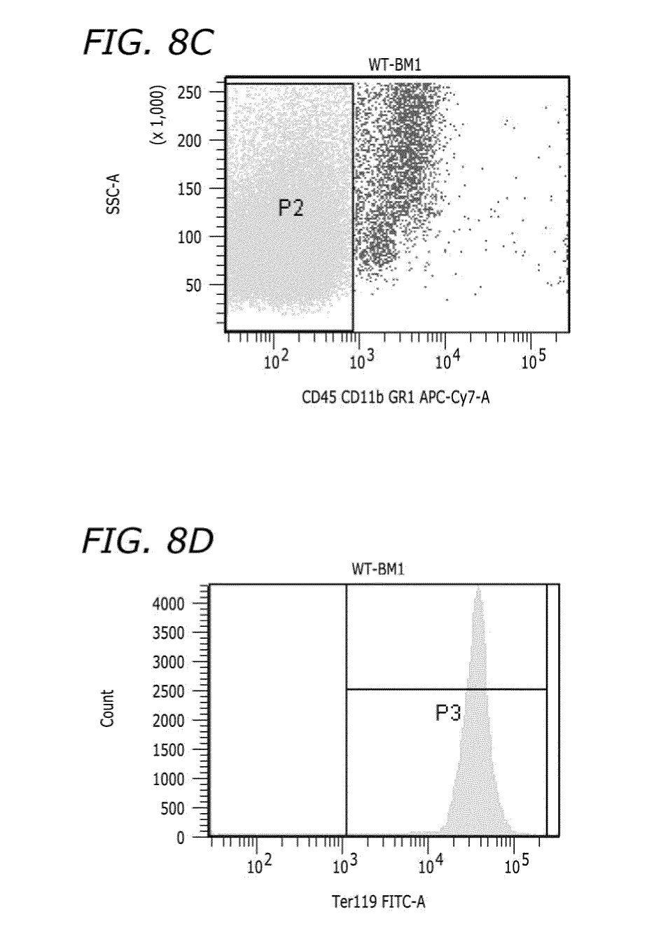

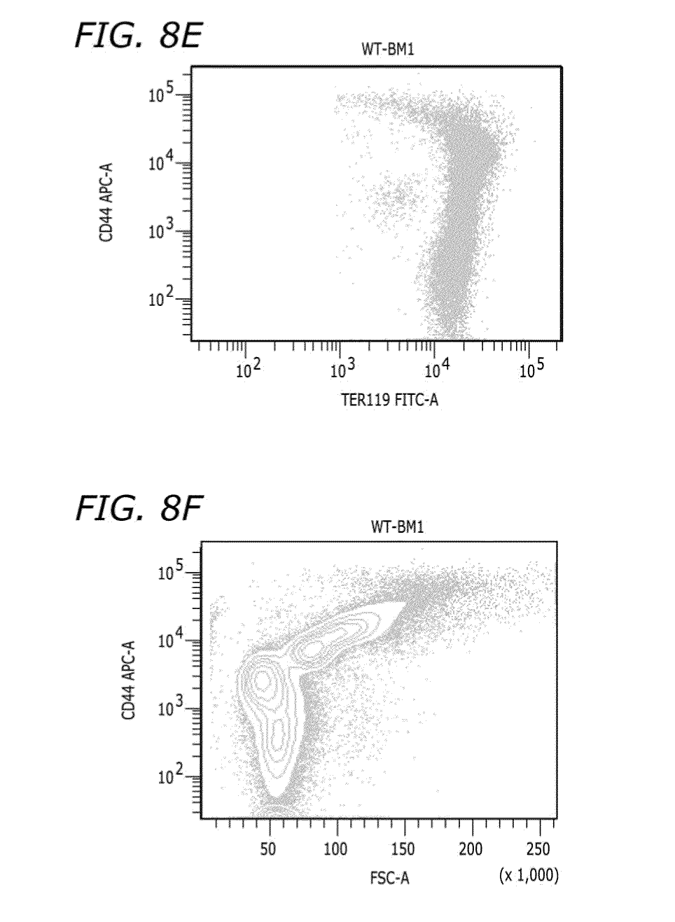

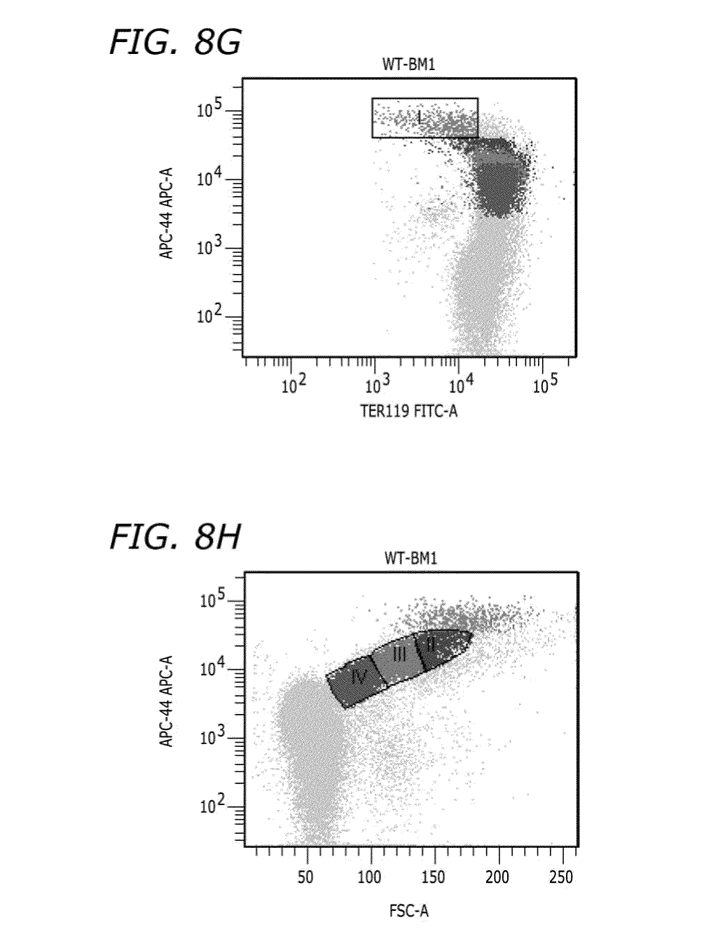

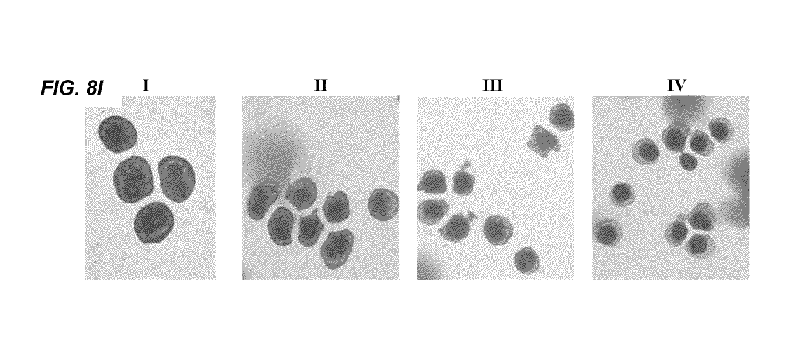

Two distinct erythroid progenitors have been functionally defined in colony assays, namely, the early-stage burst forming unit-erythroid (BFU-E), and the later stage colony forming unit-erythroid (CFU-E) progenitor. The earliest morphologically recognizable erythroblast in hematopoietic tissues is the proerythroblast, which undergoes 3 to 4 mitoses to produce reticulocytes. Morphologically distinct populations of erythroblasts are produced by each successive mitosis, beginning with proerythroblasts and followed by basophilic, polychromatic and orthochromatic erythroblasts. Based on the changes in expression levels of CD44, GPA and cell size, a method was developed to isolate populations of erythroblasts at each stage of development, in a more homogenous state than previously achieved, dependent on the expression levels of the transferrin receptor, CD71. In this study, the ratio of proerythroblast:basophilic:polychromatic:orthromatic follows the 1:2:4:8 manner, demonstrating the doubling of cells upon each mitosis. Progression of erythropoiesis is normal in 4.1R-knockout mice (a hemolytic anemia model), however in thalasemia mice the progression from proerythroblasts to basophilic erythroblasts is altered (Table 2). Thus the disclosed method now enables the quantification of in vivo erythropoiesis of mouse bone marrow and to define stage-specific defects in erythroid maturation in inherited red cell disorders. Flow cytometry of mouse bone marrow cells is depicted in FIG. 8A-8I.

TABLE 2
Quantitative Analysis of Erythropoiesis of Mouse Bone Marrow

| | WT | 4.1R KO | Thalassemia |
|---|---|---|---|
| proerythroblast | 5.4 ± 0.6 | 5.5 ± 0.4 | 2.0 ± 0.8 |
| basophilic | 13.0 ± 0.5 | 12.8 ± 0.3 | 11.7 ± 4.5 |
| polychromatic | 28.0 ± 2.0 | 27.8 ± 0.5 | 29.4 ± 1.9 |
| orthochromatic | 53.0 ± 1.7 | 53.9 ± 0.4 | 54.2 ± 1.6 |

Materials and Methods

Harvest cells. Bone marrow cells were harvested from tibia and femur of 3-month-old mice.

CD45 positive cells depletion. The cell number was determined and then the cells were centrifuged at 300×g for 10 min. The supernatant was removed and the cell pellet was resuspended in 90 μL of buffer per 10⁷ total cells. Next, 10 μL of CD45 MicroBeads were added per 10⁷ total cells and the cell suspension was mixed well and incubated for 15 min at 4-8° C. The cells were then washed by adding 1-2 mL of buffer per 10⁷ cells and centrifuged at 300×g for 10 min. The supernatant was removed and the cell pellet was resuspended at 10⁸ cells in 500 μL of buffer. This cell population was then used for magnetic separation.

Magnetic separation with MS or LS columns. Magnetic separation was conducted according to the column's manufacturer's instructions. The cells which pass through the columns were collected in a clear 15 mL tube, washed twice in 1 mL of buffer and the total effluent was collected.

Staining. 2×10⁶ cells were resuspended in 80 μL DPBS/0.5% BSA and the cells were blocked with rat anti-mouse CD16/CD32 (1 μg/10⁸ cells) for 15 min. Samples were subsequently stained with FITC rat anti-mouse TER119 (1 μg/10⁸ cells), APC rat anti-mouse CD44 (0.5 μg/10⁸ cells) and APC-Cy™ 7 rat anti-mouse CD11b (0.2 μg/10⁸ cells) on ice for 20-30 min in the dark. Cells were washed twice with 700 μL DPBS/0.5% BSA. Finally, the cells were resuspended in 100 μL DPBS/0.5% BSA and stained with the viability marker 7-AAD (0.25 vg/10⁶ cells) on ice for 10 min in the dark. The cells were then suspended in 0.4 mL of PBS/0.5% BSA and analyzed within 1 hr following staining by flow cytometry. Unstained cells were used as a negative control.

### Example 3

### Analysis of In Vitro Human Erythropoiesis

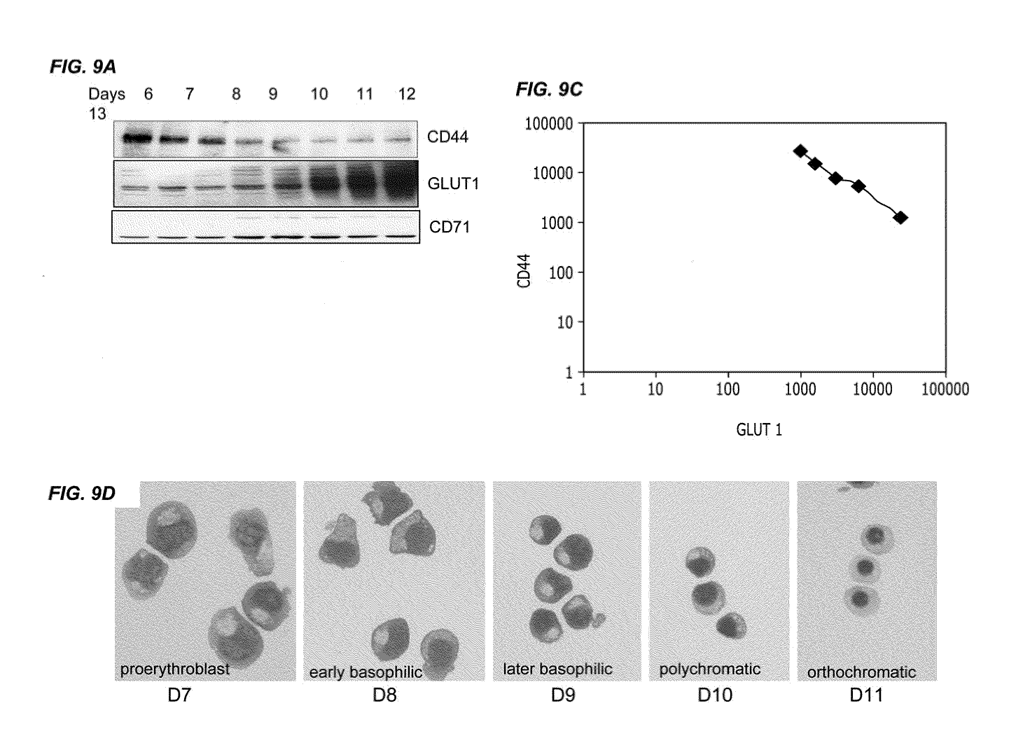

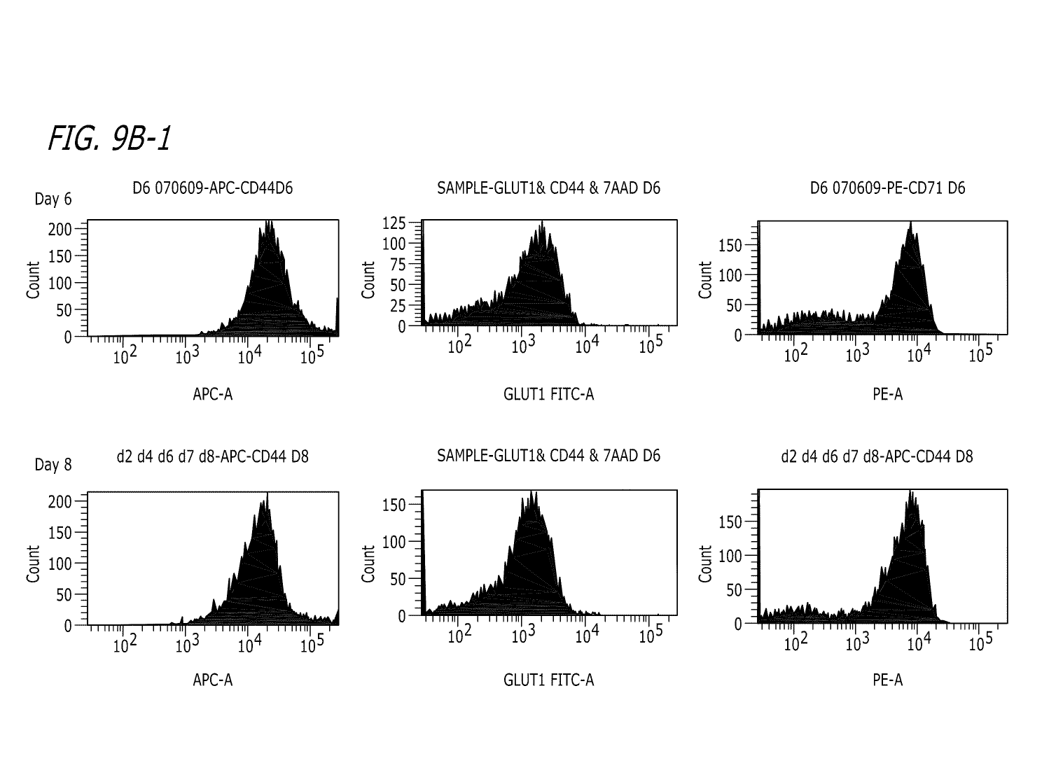

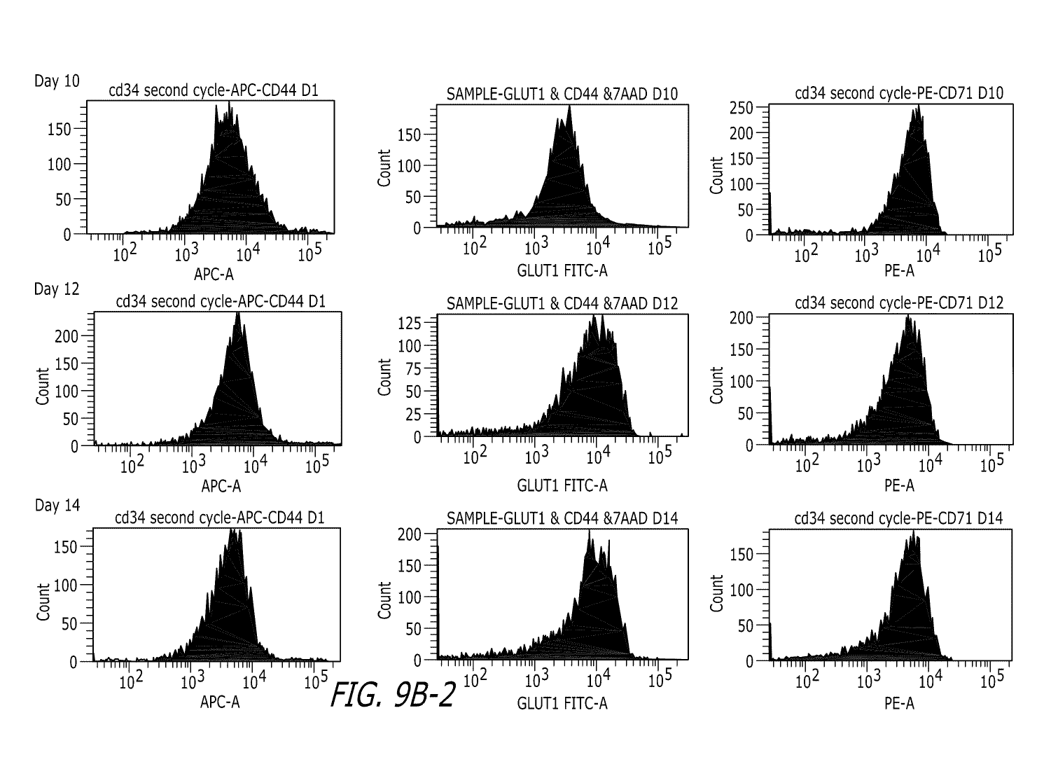

Erythropoiesis is the process by which nucleated erythroid progenitors proliferate and differentiate to generate, every second, millions of non-nucleated red cells with their unique discoid shape and membrane material properties. The time-course appearance of individual membrane protein components during murine erythropoiesis was studied and distinct changes of individual proteins were found during terminal erythroid differentiation, particularly a progressive and dramatic decrease of CD44 from proerythroblast to reticulocyte. These findings have allowed the development of a new strategy for quantifying the in vivo erythropoiesis and defining stage-specific defects in erythroid maturation in mouse. To examine whether the similar strategy can be applied to study human erythropoiesis, the expression of various red cell membrane proteins were examined during terminal differentiation of human erythroid progenitors using a human unilineage erythroid culture system. A similar pattern of membrane protein expression was noted. Particularly CD44 was decreased, GLUT1 and band 3 were increased, and CD71 (FIGS. 9A and 9B) did not exhibit obvious changes. Using CD44 and GLUT1 (or band 3) as surface markers, it was possible to isolate distinct stages of erythroblasts from in vitro cultured erythroblasts by FACS (Fluoresence Activated Cell Sorting) (FIGS. 9B and 9D). These findings strongly suggest that CD44 in conjunction with GLUT1 or band 3 can be used to distinguish erythroblasts at successive developmental stages during human erythropoiesis, which offers a means of defining stage-specific defects in erythroid maturation in inherited and acquired red cell disorders and in bone marrow failure syndromes.

Materials and Methods

Isolation of mononuclear cells using Ficoll-Paque. Core blood was diluted 2-4 volumes with buffer and the mononuclear cells were separated with Ficoll isolation by density gradient centrifugation. The interphase layer cells were transferred to a new 50 ml conical tube, washed to 4 times with buffer and resuspend in appropriate amount of buffer prior to magnetic separation.

CD34+ Magnetic Labeling. The isolated cells were spun down at 200×g for 3-5 min, 4° C. For every 1×10⁸ of cells, the cells were resuspended in 300 μl buffer and 100 μl of FcR Blocking Reagent and 100 μl of CD34 microbeads were added. The cells were mixed well and incubated at 4° C. for 30 min. The cells were then washed in 2-5 ml of buffer and resuspended in 1-2 ml of buffer.

Magnetic separation with LS columns. Magnetic separation was conducted according to the column's manufacturer's instructions. Briefly, the column was prepared by rinsing with appropriate amount of buffer and the cell suspension was applied to the column. The column was washed with 3×3 ml of buffer. The column was then removed the separator and placed in a suitable collection tube and the magnetically labeled cells were flushed from the column by firmly pushing the plunger into the column. The cells were counted and spun down at 200×g for 3-5 min at 4° C.

Cell culture. Cells were cultured by two steps protocol. In the first step (day 0-day 6), 10⁶/ml CD34+ cells were cultured (day 0) in Serum-Free Expansion Medium (SFEM, Stem Cell Technologies) supplemented with 10% fetal bovine serum (FBS), 50 ng/ml Stem Cell Factor (SCF), 10 ng/ml IL-3, 1 U/ml erythropoietin (EPO), α-thioglycerol (0.6 μl/ml medium after 1:00 dilution) and penicillin-streptomycin 100×. After 4 days in culture, the cells were diluted to 10⁶/ml using fresh medium and continue to culture three days. In the second step (day 7-day 13), cells were cultured at 10⁶/ml in SFEM medium supplemented with 30% FBS, with the same concentration of EPO, α-thioglycerol and penicillin-streptomycin.

Cell staining and sorting. 5×10⁶ cells were resuspended in 400 μl DPBS/0.5% BSA. Samples were stained with APC rat anti-mouse CD44 (0.5 μg/10⁶ cells) on ice for 20-30 min in the dark. Cells were washed twice with 700 μL DPBS/0.5% BSA. Then, cells were resuspended in 400 μL DPBS/0.5% BSA and stained with the GFP-GLUT1 antibody at 37° C. for 30 min in the dark. After washing, the cells were resuspended in 400 ml DPBS/0.5% BSA and stained with the viability marker 7-AAD (0.25 μg/10⁶ cells) on ice for 10 min in the dark. Unstained cells were used as a negative control. Single color stain sample were used as compensation. The sample then analyzed within 1 hr following staining by flow cytometry.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the invention so claimed are inherently or expressly described and enabled herein.

Furthermore, numerous references have been made to patents and printed publications throughout this specification. Each of the above-cited references and printed publications are individually incorporated herein by reference in their entirety.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

1. A method for the isolation of red blood cells (RBCS) or RBC precursors at different developmental stages comprising the steps of:
(a) obtaining a sample containing multiple maturation stages of RBCS or RBC precursors; and
(b) isolating cells that express the cell surface marker CD44.

2. The method of claim 1 further comprising determining the size of the RBCS or RBC precursors.

3. The method according to claim 2 wherein the differentiation stage of the RBC or RBC precursor is determined by CD44 expression and cell size.

4. The method according to claim 1 further comprising the step of quantifying the number of RBCS or RBC precursors in said developmental stage.

5. A method for identifying the RBC maturation stage in a disorder of RBCS.

6. The method according to claim 5 wherein the disorder of RBCS is selected from the group consisting of thalassemia, disorders of RBC maturation and bone marrow failure syndromes.

7. The method according to claim 6 wherein the disorder of RBC maturation is a myelodysplastic syndrome.

8. The method according to claim 6 wherein the thalassemia is Cooley's anemia.

9. The method according to claim 5 comprising the steps of claim 1.

10. The method according to claim 5 further comprising the step of quantifying the number of RBCS or RBC precursors in said RBC maturation stage in said disorder of RBCS.

11. A method for monitoring ex vivo proliferation and differentiation of stem cells into hematopoietic precursors and mature red cells comprising the steps of claim 1.

12. The method of claim 11 wherein said hematopoietic precursor is a red blood cell precursor.

13. The method of claim 11 further comprising the step of quantifying the number of hematopoietic precursors, RBC precursors or mature RBCS in an RBC maturation or differentiation stage.

14. The method of claim 11 wherein said stem cells are from a source selected from the group consisting of peripheral blood, bone marrow, cord blood, and placenta.

15. The method of claim 11 wherein the stem cells are embryonic stem cells.
